(19) United States
(12) Patent Application Publication
(10) Pub. No.: US 2021/0181200 A1
(43) Pub. Date: Jun. 17, 2021

(54) OVARIAN CANCER BIOMARKER AND METHODS OF USING SAME

(71) Applicant: Women's College Hospital, Toronto (CA)

(72) Inventor: Mohammad R. AKBARI, Toronto (CA)

(73) Assignee: Women's College Hospital, Toronto (CA)

(21) Appl. No.: 17/121,012

(22) Filed: Dec. 14, 2020

Related U.S. Application Data
(60) Provisional application No. 62/949,117, filed on Dec. 17, 2019.

(30) Foreign Application Priority Data
Dec. 17, 2019 (CA) 3065603

(51) Int. Cl. G01N 33/574; G01N 33/68

(52) U.S. Cl. CPC G01N 33/57449; G01N 2496/00; G01N 33/68

(57) ABSTRACT

The present invention provides a novel ovarian cancer marker, Arresten, and related methods, agents, and kits using same. The invention includes methods for detecting or diagnosing ovarian cancer, especially at early stages of the disease. The invention also includes methods of assessing the severity of ovarian cancer and monitoring responses to treatment for ovarian cancer using the biomarker.

### CROSS-REFERENCE TO RELATED APPLICATIONS

This application claims priority from, and the benefit of, U.S. Provisional Patent Application No. 62/949,117, filed Dec. 17, 2019, and Canadian Patent Application No. 3,065,603, filed Dec. 17, 2019, the entire contents of which are hereby incorporated by reference herein.

### FIELD OF INVENTION

This invention relates to a novel ovarian cancer biomarker and to related methods, uses, agents, and kits. More specifically, the invention relates to methods of diagnosing, prognosing, and treating ovarian cancer using the biomarker. The invention also relates to methods of assessing the severity of ovarian cancer and monitoring responses to treatment for ovarian cancer using the biomarker.

### BACKGROUND

Ovarian cancer remains a major health concern worldwide, accounting for 6% of all cancer deaths [1]. It is the second most common gynecological neoplasm, with over 2,800 new cases diagnosed in Canada in 2016 alone, and more than 1,800 deaths in the same year [2]. In Canada, the 5-year net survival for ovarian cancer is approximately 44%; the survival rate has only modestly increased by 2 to 4% since 1995.

Ovarian cancer is a general term for a group of neoplasms originating from the ovary, the majority (about 90%) of which are classified as epithelial carcinomas. Epithelial ovarian cancers (EOCs) comprise several morphologically distinct groups: serous, mucinous, endometrioid, clear cell, transitional cell, squamous cell, and mixed epithelial neoplasms [3].

High-grade serous ovarian cancer (HGSOC) is an aggressive subtype that accounts for more than 70% of all ovarian cancer cases and 90% of all ovarian cancer deaths. Worldwide, HGSOC is the eighth most frequent cause of cancer-related deaths in women [4]. A majority of HGSOC patients present with advanced disease and undergo surgical debulking followed by combinations of platinum drugs and paclitaxel [5]. They commonly relapse within two years and develop broad chemoresistance, leading to a very poor prognosis [6].

Over the past decade, there has been relatively little improvement in ovarian cancer survival rates. The majority of ovarian cancer cases remain asymptomatic in the early stage and only present at an advanced stage, at which point the disease is rarely curable by existing standards of care. As a consequence, ovarian cancer shows the highest mortality rate among gynecologic cancers, with only a 29% 5-year survival rate for advanced ovarian cancer. Importantly, disease outcome is significantly higher (5-year survival rates over 90%) with early diagnosis in stages I and II [7-9].

Accordingly, there exists a need for additional options, including biomarkers, for early detection of primary ovarian cancer. Such biomarkers can be useful in periodic screening of asymptomatic women for ovarian cancer and also as diagnostic tools for detecting ovarian cancer in women with a broad range of unspecific symptoms of ovarian cancer.

### SUMMARY

As described in further detail herein, the inventor has surprisingly determined that the Arresten polypeptide or a portion thereof can be used as a biomarker for cancer, particularly ovarian cancer. In conjunction with this discovery, the inventor has discerned that detection of Arresten expression can serve as a surrogate for the detection of mutated or dysfunctional p53 protein in ovarian cancer cells. These discoveries have broad implications in the diagnosis, prognosis, monitoring, and treatment of cancer, including ovarian cancers, and especially type II ovarian cancers, which are typically associated with mutated or dysfunctional p53.

Thus, in one aspect, the present application provides a method for determining whether a subject has ovarian cancer, by assaying a diagnostic sample of the subject for Arresten expression, where detection of Arresten expression elevated above normal is diagnostic of cancers, particularly ovarian cancer, in the subject. In certain embodiments, the ovarian cancer is a p53-associated ovarian cancer. In accordance with the method, Arresten expression elevated above normal can be detected, for example, by detecting p53-Arresten interaction elevated above normal. Also provided is a method of screening a general population for ovarian cancer, by testing a plurality of asymptomatic subjects in accordance with the above method.

Additionally, the present application provides a method for treating ovarian cancer in a subject, by analyzing a diagnostic sample of the subject for Arresten expression, and providing a therapy to the subject when the Arresten expression is elevated above normal. By way of example, the therapy can comprise at least one of: surgical debulking, chemotherapy, radiation, and hormone therapy. In certain embodiments, the ovarian cancer is high-grade serous ovarian cancer of stage I-IV (e.g., stage I-IIIb).

The present application also provides a method for determining the molecular subtype (type I or II) of ovarian cancer and/or for assessing the severity of the disease in a patient who has been diagnosed with ovarian cancer. The method includes assaying a biological sample of the patient for Arresten expression prior to treatment and then either: (a) determining a positive or favorable prognosis when Arresten expression in the biological sample is normal, or (b) determining a negative or poor prognosis when Arresten expression in the biological sample is elevated above normal. In accordance with the method, normal Arresten expression can be indicative of non-mutated p53 ovarian cancer, and Arresten expression elevated above normal can be indicative of mutated p53 ovarian cancer—a hallmark of molecular subtype II ovarian cancer.

The present application further provides a method for assessing the efficacy of therapy to treat ovarian cancer in a subject who has undergone or is undergoing treatment for ovarian cancer. In accordance with the method, the efficacy of the therapy may be assessed by assaying a first diagnostic sample of the subject for Arresten expression after therapy has commenced, obtaining a first level of Arresten expression in the first diagnostic sample, and then comparing the first level with a second level of Arresten expression in a second diagnostic sample of the same subject, where the second diagnostic sample was assayed and the second level was obtained prior to the therapy. A significant decrease of the first detected level, relative to the second level, can indicate that the subject is responding to the therapy to treat ovarian cancer; a minor or no decrease of the first detected level, relative to the second level, can indicate that the subject is not responding to the therapy to treat ovarian cancer. In some embodiments, the first level is below a concentration selected from 400-1000 ng/ml, and the second level is above the selected concentration.

The present application also provides a method for assessing the prognosis of a subject who has ovarian cancer, by assaying a diagnostic sample of the subject for Arresten expression. In accordance with the method, the subject's prognosis improves with a decrease in Arresten expression in the diagnostic sample, and the subject's prognosis worsens with an increase in Arresten expression in the diagnostic sample.

In the methods of the present invention described herein, the diagnostic sample (e.g., plasma, urine, etc.) may be assayed using an agent reactive with Arresten. In some embodiments, the agent is an antibody or an antigen-binding fragment thereof. The agent may also be labeled with a detectable marker. Furthermore, the diagnostic sample may be assayed using an ELISA, a chemiluminescence assay, an immunohistochemistry assay, and the like. In certain embodiments, Arresten expression is considered to be elevated above normal when the Arresten expression is above a concentration selected from 400-1000 ng/ml, preferably 446-1020 ng/ml.

Additionally, in the methods of the present invention described herein, the subject or patient may be at least one of the following: (a) pre-menopausal; (b) asymptomatic of ovarian cancer; (c) not carrying a mutation of BRCA1 or BRCA2 gene; (d) suffering from a non-malignant gynecologic disease, a peritoneal, pleural, or musculoskeletal inflammatory disorder, a pelvic inflammatory disease, a liver, renal, or cardiac disease, or an advanced adenocarcinoma. In some embodiments, the ovarian cancer is a type II ovarian cancer, particularly at stage I, II, IIIa, or IIIb. By way of example, the ovarian cancer may be undetectable by existing tests, such as CA125 test or a transvaginal ultrasound.

In some embodiments of the present invention, Arresten expression can be detected in conjunction with the detection of at least one additional biomarker, such as CA125 or HE4. In particular, expression of CA125 may be detected in addition to Arresten. An exemplary cut-off value for Arresten expression can be in the range of 400-1000 ng/ml, preferably 446-1020 ng/ml; an exemplary cut-off value for CA125 can be in the range of 95-100 U/ml.

In another aspect, the present application provides a kit for use in detecting ovarian cancer, including an agent reactive with Arresten and at least one reagent suitable for detecting expression of Arresten. The kit can be adapted or configured for ELISA-based point-of-care testing. The kit can also include a second agent reactive with another biomarker, such as CA125, and at least one second reagent suitable for detecting expression of the other biomarker.

In a further aspect, the present application provides a device for determining whether a subject has ovarian cancer, by detecting Arresten expression in a urine sample of the subject. By way of example, the device can include a housing, a matrix of absorbent material, and an immunoassay strip. In certain embodiments, the housing contains the matrix and the strip (e.g., at least partially coated with a labeled anti-Arresten antibody). In use, a urine stream or urine sample of the subject can contact the absorbent matrix or pad of the device, which draws the liquid by capillary action into the immunoassay strip. Exemplary absorbent materials for use in the device of the present invention include nitrocellulose, polysulfones, polycarboxylic acids, filter paper, and the like.

As discussed in further detail below, the present application identifies Arresten as a new biomarker for ovarian cancer, with a sensitivity and specificity that are higher than the existing biomarkers (e.g., CA125, HE4). Also described are uses of an antibody specific to Arresten for the diagnosis, treatment, or prognosis of ovarian cancer in a subject.

Using the methods, kits, antibodies, and devices of the present invention, it is possible to detect ovarian cancer, particularly type II ovarian cancer, at early stages of the disease (e.g., stage I or II). Moreover, it is possible to screen asymptomatic women periodically for ovarian cancer, and to detect ovarian cancer in women with a broad range of unspecific symptoms of ovarian cancer.

Additional aspects of the present invention will be apparent in view of the description which follows.

### BRIEF DESCRIPTION OF THE FIGURE

Embodiments of the invention will now be described, by way of example only, with reference to the accompanying drawing, in which:

FIG. 1 shows distribution of Arresten plasma level among patient cases (true) and controls (false).

### DETAILED DESCRIPTION OF THE INVENTION

Prior to the present invention, Arresten had not been investigated in the context of ovarian cancer and has never been considered as a potential serum biomarker for ovarian cancer. The existing literature did not support use of Arresten as an ovarian cancer biomarker because evidence showed that loss of p53 activity in human tumours resulted in reduced levels of Arresten [45]. The inventor's unexpected results, as disclosed herein, now identify Arresten as a new biomarker for diagnosis of ovarian cancer, especially early-stage ovarian cancer.

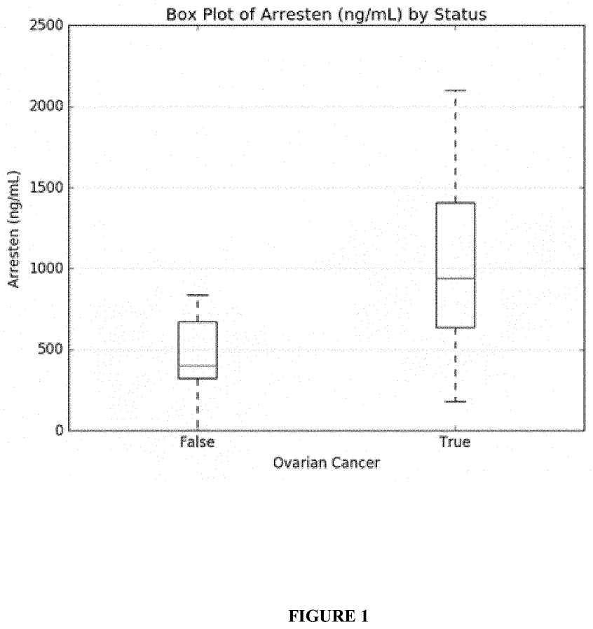

More specifically, the inventor demonstrates herein a significant increase of Arresten expression in diagnostic samples from ovarian cancer patients, as compared with healthy controls (see, e.g., FIG. 1). Accordingly, in one aspect, the present invention provides methods for determining whether a subject has ovarian cancer. The methods can include assaying a diagnostic sample of the subject for expression of Arresten, where detection of Arresten expression elevated above normal is diagnostic of neoplasia, particularly ovarian cancer, in the subject.

The Arresten biomarker disclosed herein can be used in methods for the diagnosis, prognosis, treatment, and monitoring of cancer, particularly ovarian cancer. In some embodiments, the methods of the present invention can be used to discriminate between healthy subjects and cancer subjects, including subjects with early-stage (e.g., stages I and II) disease. The methods can be based on the early detection, identification, or quantification of the Arresten biomarker, which is particularly well-suited to discriminate between healthy subjects and ovarian cancer subjects. The cancer subjects can include asymptomatic subjects and/or those at an early stage of the disease.

As a biomarker of ovarian cancer, Arresten can be detected in a biological sample, either alone or in combination with additional known biomarkers. By way of example, the biomarker can be detected, identified, or quantified by a screening method using biological or diagnostic samples from subjects. For instance, the biomarker can be detected in a blood or urine test. Known biomarkers of ovarian cancer include, without limitation, Cancer Antigen 125 (CA125) and Human Epididymis Protein 4 (HE4). Detection of Arresten expression in conjunction with detection of CA125 and/or HE4 can be particularly useful in the early detection of ovarian cancer and may significantly improve the accuracy of detecting pre-malignant changes or early-stage ovarian cancers in asymptomatic women at increased risk for the development of ovarian cancer.

Moreover, as a biomarker of ovarian cancer, Arresten can be detected and quantified in a biological sample in conjunction with other diagnostic techniques. For example, a test based on Arresten as a biomarker can be used in conjunction with vaginal examination, ultrasound, or MRI to diagnose ovarian cancer.

As noted above, ovarian cancer is asymptomatic in the early stages and most patients present with advanced levels of the disease. Cost-effective and non-invasive methods that can promote frequent testing may achieve early detection and high survival rates in ovarian cancer patients. Accordingly, in some embodiments of the methods described herein, Arresten can be detected through non-invasive tests. For example, subjects can be screened for Arresten expression using a simple and inexpensive detection module based on the well-known enzyme-linked immunosorbent assay (ELISA). This option can be useful for detecting Arresten in urine samples from subjects, including ovarian cancer patients.

Other objects, features, and advantages of the invention will become apparent from the following discussion. It should be understood, however, that the specific examples and preferred embodiments of the invention described herein are given by way of illustration only, and various changes and modifications within the scope of the invention will become apparent to those skilled in the art from the description which follows.

### Definitions

The following definitions are presented as an aid to understand the invention.

The term “subject” as used herein refers to a mammal, including, without limitation, a cow, dog, human, monkey, mouse, pig, or rat. Preferably, the subject is a human. More preferably, the subject is a woman.

The terms “sample”, “biological sample”, “diagnostic sample”, and the like, as used herein, refer to a material known or suspected of expressing or containing one or more cancer markers. The diagnostic sample may include any bodily fluids, tissues, or cells (e.g., blood, serum, plasma, urine, saliva, ovary tissues, mammary tissues, etc.). The sample is preferably a bodily fluid sample, such as blood, serum, plasma, vaginal secretions, urine, tears, saliva, etc.

As used herein, “blood” or “blood sample” can include a sample of whole blood, serum, or plasma, unless a different meaning is specified.

The terms “cancer” and “neoplasm” refer to a proliferation of tumour cells in tissue having the unique trait of loss of normal controls, resulting in unregulated growth, lack of differentiation, local tissue invasion, and/or metastasis, and include malignant tumours that are either invasive or non-invasive.

The phrase “primary cancer” refers to a cancer that is at a location of the body or a tissue where the particular cancer starts. Primary cancer is the opposite of metastasis, which refers to the migration of cancer cells from the original tumour site to produce cancer in other tissues. For example, a cancer originating in the ovary is called a “primary ovarian cancer”. If it metastasizes and spreads to the liver, the cancer is considered a primary ovarian cancer metastatic to the liver.

The terms “type I”/“subtype I” and “type II”/“subtype II” are used herein to refer to cancers, particularly ovarian cancers, which have remarkably different molecular genetic features as well as morphologic differences. For example, high-grade serous carcinoma (type II) is characterized by very frequent TP53 mutations, but rarely has mutations that characterize most type I carcinomas, including KRAS, BRAF, ERBB2, PTEN, CTNNB1, and PIK3CA. In general, type I tumours are genetically more stable than type II tumours and display a distinctive pattern of mutations that occur in specific cell types. Type II tumours which show greater morphologic and molecular homogeneity are genetically unstable and have a very high frequency of TP53 mutations. Therefore, it has been suggested that these two different types of ovarian cancers develop along different molecular pathways.

The term “marker” or “biomarker” refers to an indicator which can be detected in a sample, and includes predictive, diagnostic, and prognostic indicators and the like. The biomarker can be an indicator of a particular disease or disorder (e.g., ovarian cancer or other cancer), having certain molecular, pathological, histological, and/or clinical features.

The “presence”, “amount”, or “level” of a marker associated with an increased clinical benefit or disadvantage to an individual includes a detectable level of the marker in a sample. The presence, amount, or level of a marker can be measured by methods known to a person skilled in the art. Furthermore, the presence, amount, or level of a marker may be measured prior to treatment, during treatment, after treatment, or a combination of any of the foregoing.

As used herein in connection with Arresten, the term “expression” includes, without limitation, the transcription of the Arresten-associated gene into at least one mRNA transcript and or the translation of at least one mRNA transcript into an Arresten protein. Accordingly, a diagnostic sample may be assayed for Arresten expression by assaying for Arresten, Arresten cDNA, or Arresten mRNA. The appropriate form of Arresten will be apparent based on the particular techniques discussed herein.

The phrase “elevated above normal”, as used herein, refers to expression of Arresten (or p53-Arresten interaction) that is detected at a level significantly greater than the level expected for the same type of diagnostic sample taken from a non-diseased subject or patient (i.e., one who does not have cancer, such as ovarian cancer) of the same gender and of similar age. As further used herein, “significantly greater” refers to a statistically significant difference between the level of Arresten expression elevated above normal and the expected (normal) level of Arresten. Preferably, Arresten expression that is elevated above normal is expression of Arresten at a level that is at least 10% greater than the level of Arresten expression otherwise expected. Where Arresten expression is expected to be absent from a particular diagnostic sample taken from a particular subject or patient, the normal level of Arresten expression for that subject or patient is nil. Where a particular diagnostic sample taken from a particular subject or patient is expected to have a low level of constitutive Arresten expression, that low level is the normal level of Arresten expression for that subject or patient. As disclosed herein, Arresten-p53 interactions, and Arresten expression, are generally present at low levels in normal ovary cells, including those that do not contain dysfunctional p53 protein.

A “reference sample” or “control sample”, as discussed herein, is a biological sample provided from a reference or control group of apparently healthy individuals for the purpose of evaluation in vitro. Similarly, the expressions “reference concentration”, “reference value”, and “reference level”, as used herein, refer to a value established in a reference or control group of apparently healthy individuals. Determination of the reference concentration of Arresten or Arresten expression can be made based on an amount or concentration which best distinguishes patient and healthy populations. By way of example, the value for Arresten as determined in a control group or a control population establishes a “cut-off value” or a “reference range”. A value above such cut-off or threshold, or outside the reference range at its higher end, is considered to be “elevated above normal” or “diagnostic of ovarian cancer”. The reference level can be a single number, equally applicable to every subject, or the reference level can vary, according to specific subpopulations of subjects. For example, post-menopausal subjects can have a different reference level for ovarian cancer than pre-menopausal subjects. In addition, a subject with more advanced ovarian cancer (e.g., stages II-IV) can have a different reference value than one who has early stage ovarian cancer (e.g., stage I).

As used herein, an agent “reactive” with Arresten is one that has affinity for, binds to, or is directed against Arresten. Such an “agent” can be a protein, polypeptide, peptide, nucleic acid (including DNA or RNA), antibody, Fab fragment, F(ab′)₂ fragment, molecule, compound, antibiotic, drug, or any combination thereof. A Fab fragment is a univalent antigen-binding fragment of an antibody, which is produced by papain digestion. A F(ab′)₂ fragment is a divalent antigen-binding fragment of an antibody, which is produced by pepsin digestion. Preferably, the agent of the present invention is labeled with a detectable marker or label.

The term “antibody”, as used herein, refers to a specific protein molecule directed against an antigenic site and broadly includes all different types of antibody structures, such as monoclonal antibodies, polyclonal antibodies, multi specific antibodies (including bispecific antibodies), chimeric antibodies, humanized antibodies, fragments having antigen-binding activity, etc. For purposes of the present invention, the antibody can specifically bind to the biomarkers of the present invention, or the constituent proteins of the biomarkers, and can include polyclonal antibodies, monoclonal antibodies, and recombinant antibodies. The production of antibodies—using, for example, Arresten as an antigen—can be performed with techniques well known to a person of ordinary skill in the art.

The term “label” refers to a detectable compound or composition and “labelling” refers to the conjugation, fusion, or attachment of a detectable compound or composition to another. In some embodiments, the label is conjugated or fused directly or indirectly to an agent or reagent, such as an antibody, and assists with the detection of the agent to which it is conjugated or fused. The label itself can also be detectable (such as radioisotope labels or fluorescent labels and the like). By way of example, the label may be an enzymatic label which catalyzes chemical alteration of a substrate compound or composition and results in a detectable product.

The “sensitivity” of a biomarker, test, or assay, as described herein, means the probability that the biomarker, test, or assay will yield a positive result in an individual afflicted with cancer, particularly ovarian cancer. The “specificity” of a biomarker, test, or assay, as described herein, means the probability that the biomarker, test, or assay will yield a negative result in an individual not afflicted with cancer, particularly ovarian cancer.

The term “diagnosis”, as used herein, refers to the identification or classification of a molecular or pathological state, disease, or condition (e.g., cancer, a particular type of cancer, etc.). “Diagnosis” also refers herein to the classification of a particular subtype of cancer, such as by histopathological criteria or by molecular features (including a subtype characterized by expression of one or a combination of biomarkers, such as particular genes or proteins encoded by the genes).

The term “prognosis”, as used herein, refers to the prediction of the likelihood of cancer-attributable death or progression, including recurrence, metastatic spread, and drug resistance, of a neoplasm, such as ovarian cancer. Prognosis may also be referred to in terms of “aggressiveness” or “severity”: an aggressive cancer is determined to have a high risk of negative outcome (i.e., negative or poor prognosis) and a non-aggressive cancer has a low risk of negative outcome (i.e., positive or favorable prognosis). An “aggressive” or “severe” tumour is a cell-proliferation disorder that has the biological capability to rapidly spread outside of its primary location or organ. Indicators of tumour aggressiveness that are standard in the art include, without limitation, tumour stage, tumour grade, Gleason grade, Gleason score, nodal status, and survival. In this context, the term “survival” is not limited to mean survival until mortality (wherein said mortality may be either irrespective of cause or related to a cell-proliferation disorder), but may also used in combination with other terms to define clinical outcomes (e.g., “recurrence-free survival”, in which the term “recurrence” includes both localized and distant recurrence; “metastasis-free survival”; “disease-free survival”, in which the term “disease” includes cancer and diseases associated therewith). The length of the survival may be calculated by reference to a defined starting point (e.g., time of diagnosis or start of treatment) and a defined end point.

The terms “treatment”, “treat”, “treating”, and “therapy” all refer to clinical intervention in an attempt to alter the natural course of an individual being treated, and can be performed either for prophylaxis or during the course of clinical pathology. Effects of treatment or therapy can include preventing occurrence or recurrence of disease, alleviating symptoms, diminishing any direct or indirect pathological consequences of disease, preventing metastasis, decreasing the rate of disease progression, ameliorating the disease state, minimizing the clinical impairment or symptoms resulting from the disease, diminishing any pain or discomfort suffered by the subject, remission or improved prognosis, and extending the survival of a subject beyond that which would otherwise be expected in the absence of such treatment. With reference to cancer, “treatment” and “therapy” also include inhibiting or preventing the development or spread of the cancerous cells (e.g., by limiting, suspending, terminating, or otherwise controlling the maturation and proliferation of cells involved in the cancer).

Ovarian Cancer and Diagnosis

According to conventional thought, low-grade serous carcinoma is a precursor lesion for high-grade serous carcinoma. However, recent studies support the inventor's conclusion that ovarian carcinoma has two subsets that are molecularly distinct as separate diseases characterized by differing patterns of genomic variation and prognostic implications. Low-grade serous carcinoma (e.g., classified as type I ovarian cancer) has better prognosis and a more indolent/stable genomic profile, while high-grade serous carcinoma (e.g., classified as type II ovarian cancer) has worse prognosis and an aggressive/distinct genomic profile.

Molecularly, type I ovarian cancer (OC) is often characterized by mutations in the mitogen-activated protein kinase (MAPK) pathway (e.g., KRAS or BRAF). Other significant variants for type I OC can include alterations of genes encoding β-catenin (e.g., CTNNB1), CDKN2A, PIK3CA, and PTEN which have also been found in a number of studies of type I OC. TP53 mutations have been rarely seen in type I OC, except in mucinous carcinoma.

Type II OC is characterized by a high degree of genomic/chromosomal instability, including nearly ubiquitous mutations in TP53 that arise mainly from the fallopian tube epithelium. The resulting dysfunctional p53 protein or pathway is the hallmark for the type II OC patient group. These tumours are clinically undetectable in stage I or II, and progress rapidly through stages III/IV.

Diagnosis of ovarian cancer can be performed when symptoms such as abdominal pain (bloating), gastrointestinal (e.g., bowel) irregularities), and pelvic pain (pressure or discomfort) are presented in a subject at advanced stages of the disease. 30% of patients experience a delay in diagnosis of 2-6 months. Prior to the present invention, OC has been diagnosed through a positive screening test, such as a CA125 test or transvaginal ultrasounds (TVS). Ovarian cancer can also be diagnosed incidentally after preventive surgery (i.e., for BRCA carriers).

Macroscopic residual disease after debulking surgery can be an independent prognostic factor for survival. Among women who had undergone primary debulking surgery, those with no residual disease had much better seven-year survival than women who had any residual disease (73.6% versus 21.0%; p<0.0001) [48]). Ten-year survival can be much improved when surgery results in no residual disease for stage III/IV HGSOC (50% versus 15% for any residual disease) [49].

In OC screening, the objective is to identify an ovarian cancer at a time when a cure is likely (e.g., when it is possible to achieve no residual disease after debulking). The likelihood of achieving no residual disease diminishes with the extent of abdominal disease the surgeon must remove. Therefore, among women with stage III ovarian cancer, it is desirable to diagnose cancer as early as possible with the goal of minimizing the intra-abdominal tumour burden. In one study, a status of no residual disease post-surgery was achieved for most patients with stage IIIa/b ovarian cancer, while only about 45% of patients with stage IIIc/IV ovarian cancer were reduced to no residual disease [48].

At present, 90% of patients are diagnosed in stage IIIc/IV; only 10% are diagnosed in stage IIIa/b. In this situation, a shift of patients from stage IIIc/IV to stage IIIa/b has the potential to increase the probability of cure. Currently, it is almost impossible to detect type II OCs at their stage I/II. Most stage I/II OCs that are diagnosed are type I OCs.

Existing Ovarian Cancer Biomarkers

To date, none of the known ovarian cancer serological biomarkers has been shown to be useful as a screening test in asymptomatic women because they have poor specificity and sensitivity in patients with early stage ovarian cancer. The only clinical significance of current ovarian cancer biomarkers is to differentiate between benign and malignant pelvic masses.

Current clinical guidelines recommend the use of biomarker CA125 for early triage of women with pelvic masses and for the management of patients with epithelial ovarian/fallopian tube cancer in monitoring response to first-line chemotherapy and post-therapy surveillance [10]. Concentrations of CA125 greater than 95 kU/L have discriminatory potential for malignancy in the pelvic mass, with a positive predictive value of 95% [11]. Persistent elevated levels of CA125 are indicative of a poor prognosis [12].

Nevertheless, as discussed above, the specificity and sensitivity of CA125 are far from ideal. Among epithelial ovarian cancer patients, only 80% or so have concentrations of CA125 above the reference interval of 35 kU/L. Elevations have been >90% in stages and 80-90% in stage II, but only 50-60% in patients with stage I OC [13,14].

Specificity of CA125 is compromised by its overexpression in healthy pre-menopausal women during menses and in pregnancy, as well as in some non-malignant gynecologic diseases (e.g., ovarian cysts, endometriosis, adenomyosis, uterine leiomyomas), peritoneal, pleural, and musculoskeletal inflammatory disorders, pelvic inflammatory diseases, and liver, renal, and cardiac diseases. Additionally, elevated concentrations can occur in most types of advanced adenocarcinomas, including breast, colorectal, pancreas, lung, endometrium, and cervix [14,15].

The frequency of CA125 overexpression is highest, though, in serous epithelial ovarian cancer patients, followed by endometrioid and clear cell types. CA125 is not expressed in pure mucinous histological type of epithelial ovarian cancer [14,15].

HE4 is also found to be overexpressed in ovarian cancer [16]. It has been shown to have greater specificity compared with CA125, especially in the pre-menopausal population, in which HE4 levels are less elevated than CA125 (8% versus 29% in benign pelvic mass); however, HE4 still shows varying results for sensitivity [17]. HE4 is not elevated in benign gynecological conditions (e.g., pregnancy, menstruation, endometriosis) [18].

HE4 demonstrates the highest sensitivity for stage I diagnosis (45.9% sensitivity at 95% specificity) and performs better than CA125 as an indicator of worse prognosis in epithelial ovarian cancer [19]. The HE4 serum levels in healthy women range from 60 pmol/L to 150 pmol/L, with higher serum levels observed in women over 40 years of age [20,21]. Concentrations of HE4 and CA125 are the highest in endometrioid cancer (100% overexpression) and serous epithelial ovarian cancer (93% overexpression), and the lowest in patients with mucinous ovarian carcinomas [22].

HE4 has also been identified in pulmonary, endometrial, and breast carcinomas, and mesotheliomas, but less frequently in gastrointestinal, renal, and transitional cell carcinomas. The most significant source of false-positive results in serum is renal failure [23].

In 2008, Moore et al. developed the Risk of Ovarian Malignancy Algorithm (ROMA) that uses a combination of CA125, HE4, and menopausal status to predict the presence of a malignant ovarian tumour with expected higher sensitivity and specificity compared with CA125 alone [19,24]. Several independent prospective studies and meta-analysis have been carried out in order to validate the diagnostic performance of ROMA, but they failed to reach a clear consensus.

Van Gorp et al. performed prospective validation on 389 patients and found that CA125 over-performed HE4 in post-menopausal women and neither HE4 nor ROMA improved the diagnosis of ovarian cancer [25]. There is some limited meta-analysis-based evidence of ROMA performing better in early ovarian cancer and the post-menopausal population. Other meta-analysis studies found ROMA to have higher sensitivity, but HE4 to be more specific. Overall, existing meta-analysis results do not provide strong evidence for ROMA superiority over CA125 alone [26-29].

In 2016, the United States Food and Drug Administration (FDA) approved a new-generation pre-operative serum biomarker test for ovarian cancer: the OVA1® test (Overa®). OVA1 combines 5 individual markers: CA125-II, HE4, apolipoprotein A-1, follicle stimulating hormone, and transferrin. Only after an ovarian mass has been determined to require surgery is this test then used to assess the likelihood of malignancy; thus, the use of the OVA1 test is very limited. OVA1 maintains a higher diagnostic sensitivity and high negative predictive value because five biomarkers are applied in parallel in the same serum specimen. If the test shows low-risk, the tumour is very unlikely to be malignant and the surgery can be scheduled without consulting a specialist [30].

In conclusion, CA125 is currently the only biomarker approved for routine use in ovarian cancer diagnosis. HE4, reporting increased specificity as compared to CA125, requires further validation of clinical utility. All of these biomarkers show elevation in the late-stage disease population, but it may not be clinically relevant. Since very high specificities and sensitivities are required in screening for diseases of low prevalence, neither CA125 nor HE4 qualifies as a screening marker.

Limits to Optimizing Cut-Off for Cancer Antigen 125

Cancer Antigen 125 (CA125) is expressed on the surface of ovarian cancer cells and plays a role in progression and metastasis of the disease. CA125 is also expressed in normal epithelia of the peritoneum, in endometrium, or in benign ovarian cysts. Various research has been conducted to assess the levels of CA125 in different population groups. It was reported that 1% of 883 healthy women, 6% of 143 patients with benign pelvic disease, and 82% of 102 patients with ovarian cancer showed levels of CA125 over 35 U/ml [50]. In addition, it was reported that 10% of benign pelvic disease (mostly pre-menopausal), 25% of borderline OC tumours, 40% of stage I OC tumours, and 80% of stage II-IV OC tumours showed levels more than 100 U/ml of CA125 [51].

It was previously reported that, among healthy individuals, 5% showed values of CA125 over 35 U/ml, 1% showed values over 65 U/ml, and 0.1% showed values over 100 U/ml (seen only in pre-menopausal women) [52]. More recently, Kotsopoulos et al. measured the levels of CA125 of 422 patients with ovarian cancers at different disease stages and obtained the following results [unpublished; included here with permission]:

| | | | | | | Missing/ |
|---|---|---|---|---|---|---|
| Stage | CA125, mean (range) | | Median | CA125 > 100, n (%)¹ | | No CA125, n |
| IA (n = 47) | 424.2 | (6.0-5642.0) | 73.0 | 16 | (52%) | 8 |
| IB (n = 3) | 23.7 | (9.0-42.0) | 20.0 | 0 | | 0 |
| IC (n = 44) | 1013.6 | (2.0-40542.0) | 54.0 | 15 | (38%) | 5 |
| IIA (n = 17) | 224.9 | (8.0-1298.0) | 114.0 | 10 | (63%) | 1 |
| IIB (n = 28) | 556.3 | (14.0-2912.0) | 184.0 | 16 | (62%) | 2 |
| IIIA (n = 16) | 553.7 | (15.0-2191.0) | 310.0 | 12 | (80%) | 1 |
| IIIB (n = 45) | 1191.2 | (27.0-12992.0) | 516.0 | 36 | (84%) | 2 |
| IIIC (n = 145) | 1864.2 | (2.0-26056.0) | 719.5 | 125 | (87%) | 1 |
| IV (n = 57) | 1897.2 | (54.0-11835.0) | 762.5 | 53 | (98%) | 3 |
| Missing (n = 20) | 1871.2 | (4.0-14450.0) | 199.0 | 13 | (81%) | 4 |

As the above data show, if the cut-off value for CA125 is changed from 35 to 100 kU/L, more than 80% of ovarian cancer patients with stages III and IV will be identified; however, patients with stage I/II may not be identified.

As discussed above, the main limitations to the use of CA125 as a biomarker for ovarian cancer are its low sensitivity for stage I/II and its low specificity for pre-menopausal women. Currently, the recommended cut-off for CA125 is 35 U/ml internationally. Raising the cut-off to 100 U/ml could address both of these limitations, but at the expense of missing borderline and stage I/II tumours, which are biologically low-risk and have a 10-year survival of over 80%.

p53 and Arresten in Oncogenesis

p53 is a tumour suppressor and the most frequently mutated gene in human malignancy, with mutations identified in at least 50% of human cancers [31-33]. In the wild-type state, p53 promotes processes consistent with tumour suppression, including cell cycle inhibition, apoptosis, senescence, DNA repair, autophagy, and metabolic processes that prevent oncogenic reprogramming [34]. Pathogenic mutations of p53 result in loss of tumour suppressive functions, mostly by affecting p53 binding to the response elements in DNA and disabling transcription factor activity. Along with the loss of wild-type function, many p53 mutations result in a gain of oncogenic function by accumulating in cells at high levels and contributing to tumorigenesis and development of drug resistance [35,36].

Loss of p53 function is a common event in tumour progression and has been associated with poor prognosis in ovarian carcinoma. Next to mutated p53, defects in retinoblastoma (RB) pathway, and activation of oncogenes such as c-myc, K-ras, and Akt, constitute mutational backgrounds in sporadic epithelial ovarian cancer (EOC) [37,38]. On the other side, the most common genetic alterations in hereditary ovarian carcinomas are mutations in BRCA1 and BRCA2 genes [39-41].

Type IV collagen proteins are major structural components of basement membranes. The general term “matrikines” has been employed for all collagen-derived fragments, out of which several are known to possess anti-angiogenic activity [42].

Arresten is the 26-kDa biologically active molecule and product of proteolytic cleavage derived from non-collagenous domain of type IV collagen, alpha-1 chain (encoded by the COL4A1 gene). The base sequence of the human COL4A1 gene and the amino acid sequence of the encoded protein are known. For example, the nucleotide sequence of the human COL4A1 gene and the protein amino acid sequence are registered and published in GenBank (GenBank Accession No. NM_001845). Arresten inhibits endothelial cell proliferation, at least in part, by inducing apoptotic mechanism [43].

p53 is known to induce degradation of collagen IV alpha-1 in the extracellular matrix by activating a matrix metalloproteinase-dependent mechanism, which results in release of Arresten to the blood. Recently, in human prostate tumour samples, Teodoro et al. showed for the first time that production of Arresten is highly induced in response to p53 tumour suppressor pathway [44]. p53 was shown to modulate Arresten levels through: 1) direct transcriptional stimulation of COL4A1 gene; 2) indirect mechanism of up-regulation of alpha(II)PH, a characterized target of p53, that potentiates the production of full-length alpha-1 collagen IV; and 3) promotion of the metalloproteinase-dependent proteolytic remodeling of the collagen IV matrix and subsequent enhancing of collagen processing. The consequences of this link can be observed in vivo with upregulation of alpha(II)PH that significantly inhibits tumour growth.

Therefore, mutated p53 correlates with the lack of Arresten within human prostate tumour samples. Both in vivo and in vitro data strongly support a model of p53-dependent stimulation of Arresten and consequential inhibition of angiogenesis, tumour growth, and development of metastasis [44,45]. High frequency of p53 mutation is also strongly associated with ovarian cancer, especially HGSOC, where a mutant form of p53 is detected in over 96% of cases. A body of literature supports a model in which p53 mutation constitutes an early event in the pathogenesis of HGSOC, in addition to its known role in carcinogenesis [46,47].

Arresten as a Biomarker for Ovarian Cancer

Inactivation of p53 is the hallmark of high-grade serous ovarian cancers, and almost all of these tumours have dysfunctional p53 protein. However, it is currently impractical to use mutant or dysfunctional p53 as a diagnostic or prognostic biomarker in ovarian cancer. p53 is generally not a stable protein; it has a short half-life, and is degraded very rapidly. To date, no appropriate detection protocols have been developed for mutant or dysfunctional p53.

As described herein, the inventor has identified and demonstrated that a specific protein can act as a surrogate for mutant or dysfunctional p53. Surprisingly, that surrogate protein is Arresten, the expression level of which the existing literature had shown to decrease in human plasma when mutated p53 increases in tumour cells. As shown by the inventor, while Arresten is released by p53 to the blood and p53 is dysfunctional in ovarian cancers, the level of Arresten is unexpectedly much higher in the blood of ovarian cancer patients. In contrast to the existing evidence that loss of p53 activity in ovarian tumours results in reduced levels of Arresten [45], the inventor found that the level of Arresten in ovarian cancer patients is two-fold higher in comparison to that in healthy subjects (see, e.g., FIG. 1). It is theorized that the level of Arresten increases in ovarian cancer patients because of overproduction of dysfunctional p53 protein in their ovarian cancer cells.

Prior to the present invention, there was no study carried out regarding concentration of Arresten in blood, especially in connection with early detection or diagnosis of ovarian cancer. The inventor found that Arresten was more effective than CA125 as a biomarker in ovarian cancer and its level distinguished an ovarian cancer patient from a normal subject. In other words, the sensitivity and specificity of Arresten as an ovarian cancer marker are much higher than those of CA125. Based on the teachings in the existing literature regarding the functions of Arresten, lower concentrations of the molecule were expected when p53 was dysfunctional. However, the inventor observed the opposite results in p53 ovarian cancer patients with poor prognosis: all of them showed much higher levels of Arresten in the blood as compared to normal subjects.

It may be that when p53 becomes dysfunctional in ovarian tumours, negative regulatory mechanisms induce higher expression of p53 because p53 no longer carries out the function it is supposed to do. Increased expression of dysfunctional p53 protein may still induce expression of some proteins downstream, including Arresten in ovarian tumours, and this may explain why the level of Arresten actually increases in ovarian cancer patients.

The present application identifies Arresten as an ovarian cancer biomarker that has significantly discriminative power, without compromising on sensitivity, prior to manifestation of symptoms. Therefore, it is now possible to develop diagnostic methods and tools based on the use of Arresten as a biomarker for ovarian cancer.

As is well known in the art, reference levels of tumour markers initially arise from the need for a differentiation diagnosis between patients and healthy population. Similarly, serum levels of Arresten that differentiate ovarian cancer patients from healthy controls can be determined. In one embodiment, a threshold value of Arresten that is determined to be elevated above normal (e.g., a reference concentration or cut-off value of Arresten) may be determined to be a value from 400 to 1000 ng/ml. For example, the threshold value of Arresten may be one of the following concentrations in the subject's sample (e.g., serum or plasma sample): 400 ng/ml, 450 ng/ml, 500 ng/ml, 550 ng/ml, 600 ng/ml, 650 ng/ml, 700 ng/ml, 750 ng/ml, 800 ng/ml, 850 ng/ml, 900 ng/ml, 950 ng/ml, or 1000 ng/ml. The reference concentration may also be selected from the ranges of 400-1000 ng/ml, 450-1000 ng/ml, 500-1000 ng/ml, 550-1000 ng/ml, 650-1000 ng/ml, 700-1000 ng/ml, 750-1000 ng/ml, 800-1000 ng/ml, 850-1000 ng/ml, and 900-1000 ng/ml. In certain embodiments, the reference concentration is further selected from the ranges of 400-1000 ng/ml, 400-950 ng/ml, 400-900 ng/ml, 400-850 ng/ml, 400-800 ng/ml, 400-750 ng/ml, 400-700 ng/ml, 400-650 ng/ml, 400-600 ng/ml, 400-550 ng/ml, and 400-500 ng/ml, or from the ranges of 400-1000 ng/ml, 450-950 ng/ml, 500-900 ng/ml, 550-850 ng/ml, 600-800 ng/ml, and 650-750 ng/ml. In another embodiment, the reference concentration may be about two times higher than the average concentration of Arresten in the samples of controls (e.g., serum or plasma samples). In yet another embodiment, the reference concentration may be 1.3-2.5 times, preferably, 1.5-2 times, higher than the average concentration of Arresten in the samples of controls (e.g., serum or plasma samples).

In addition, the following factors can also be determined: (i) plasma levels of Arresten, CA125, and HE4 in ovarian cancer patients at the time of diagnosis and in healthy women matched for menopause status; (ii) sensitivity and specificity of Arresten in distinguishing ovarian cancer cases from healthy controls; (iii) whether Arresten has a potential to enhance specificity and sensitivity of existing biomarkers, including CA125 and HE4, when used in combination with them; and (iv) expression levels of p53 and Arresten in ovarian tumour tissues.

As one preferred approach, Arresten can be combined with different biomarkers, such as CA125 and HE4, in the detection, diagnosis, monitoring, and prognosis of ovarian cancer. In particular, Arresten can address gaps in screening, early detection, and diagnosis of ovarian cancer. Since Arresten is cleared from blood by the kidney, a simple urine test for early detection of the disease can be provided for use by women in the comfort of their own homes (e.g., for use periodically or if they present with a symptom associated with ovarian cancer). As a consequence, Arresten as a biomarker can be used in screening methods and tools to screen the general population for ovarian cancer. Furthermore, interaction between p53 and Arresten can be characterized and quantified based on their expression levels in ovarian tumour tissues. Plasma levels of Arresten can be correlated with disease development in ovarian cancer patients, thereby providing new and non-invasive screening and diagnostic tests.

Diagnostic Samples

In accordance with the methods described herein, a diagnostic sample from the subject may be obtained using standard procedures. The diagnostic sample may be tissue and may be removed by standard biopsy. By way of example, the diagnostic sample may be any tissue known to have a neoplasm, any tissue suspected of having a neoplasm, or any tissue believed not to have a neoplasm. In addition, the diagnostic sample may be a bodily fluid, including blood, serum, plasma, vaginal secretions, urine, tears, and saliva.

Protein (e.g., Arresten) may be isolated and purified from the diagnostic sample of the present invention using standard methods known in the art, including, without limitation, extraction from a tissue (e.g., with a detergent that solubilizes the protein) where necessary, followed by affinity purification on a column, chromatography (e.g., FTLC and HPLC), immunoprecipitation (with an antibody to Arresten), and precipitation. Isolation and purification of the protein may be followed by electrophoresis (e.g., on an SDS-polyacrylamide gel). In accordance with the methods of the present invention, ovarian cancer in a subject may be diagnosed by assaying a diagnostic sample of the subject for expression of Arresten, wherein expression of Arresten elevated above normal is diagnostic of ovarian cancer.

Detecting Arresten Expression

In the methods of the present invention, a diagnostic sample of a subject may be assayed for Arresten expression, and Arresten expression may be detected in a diagnostic sample, using assays and detection methods readily determined from the known art (e.g., immunological techniques, hybridization analysis, fluorescence imaging techniques, and/or radiation detection, etc.), as well as any assays and detection methods disclosed herein (e.g., immunoprecipitation, Western blot analysis, etc.). For example, a diagnostic sample of a subject may be assayed for Arresten expression using an agent reactive with Arresten (e.g., antibody specific to Arresten).

The antibodies as used herein may be labeled with a detectable marker or label. Labeling of an antibody may be accomplished using one of a variety of labeling techniques, including a chemical (e.g., biotin), an enzyme (e.g., horseradish peroxidase, alkaline phosphatase), a radioactive material, a luminescent material, or a chemiluminescent material known in the art. For example, a nonradioactive or fluorescent marker, such as biotin, fluorescein (FITC), acridine, cholesterol, or carboxy-X-rhodamine, which can be detected using fluorescence and other imaging techniques readily known in the art. Alternatively, the detectable marker or label may be a radioactive marker, including, for example, a radioisotope. The radioisotope may be any isotope that emits detectable radiation, such as ³H, ¹⁴C, ³²P, ³⁵S, ⁴⁵Ca, or ¹²⁵I Radioactivity emitted by the radioisotope can be detected by techniques well known in the art. For example, gamma emission from the radioisotope may be detected using gamma imaging techniques, particularly scintigraphic imaging. Preferably, the agent of the present invention is a high-affinity antibody labeled with a detectable marker or label.

Where the agent of the present invention is an antibody reactive with Arresten, a diagnostic sample taken from the subject may be purified by passage through an affinity column which contains Arresten antibody as a ligand attached to a solid support, such as an insoluble organic polymer in the form of a bead, gel, or plate. The antibody attached to the solid support may be used in the form of a column. Examples of suitable solid supports include, without limitation, agarose, cellulose, dextran, polyacrylamide, polystyrene, sepharose, or other insoluble organic polymers. The Arresten antibody may be further attached to the solid support through a spacer molecule, if desired. Appropriate binding conditions (e.g., temperature, pH, and salt concentration) for ensuring binding of the agent and the antibody may be readily determined by the skilled artisan. In one embodiment, the Arresten antibody is attached to a sepharose column, such as Sepharose 4B.

Where the agent is an antibody, a diagnostic sample of the subject may be assayed for Arresten expression using binding studies that utilize one or more antibodies immunoreactive with Arresten, along with standard immunological detection techniques. For example, the Arresten molecule eluted from the affinity column may be subjected to an ELISA assay, Western blot analysis, flow cytometry, or any other immunostaining method employing an antigen-antibody interaction.

The detection of Arresten expression in the method of the present invention may be followed by an assay to measure or quantify the extent of Arresten expression in a diagnostic sample of a subject. Such assays are well known to one of skill in the art, and may include, without limitation, immunohistochemistry/immunocytochemistry, flow cytometry, mass spectroscopy, Western blot analysis, or an ELISA for measuring amounts of Arresten molecule. For example, to use an immunohistochemistry assay, histological (paraffin-embedded) sections of tissue may be placed on slides, and then incubated with an antibody against Arresten. The slides then may be incubated with a second antibody (against the primary antibody), which is tagged to a dye or other colorimetric system (e.g., a fluorochrome, a radioactive agent, or an agent having high electron-scanning capacity), to permit visualization of Arresten present in the sections.

It is contemplated that the diagnostic sample may be assayed for expression of any or all forms of Arresten protein (including precursor, endoproteolytically-processed forms, and other forms resulting from post-translational modification) in order to determine whether a subject or patient has ovarian cancer. It is also contemplated that the diagnostic sample may be assayed for expression of Arresten elevated above normal by detecting an increase in p53-Arresten interaction. Accordingly, in one embodiment of the present invention, Arresten expression elevated above normal is detected by detecting p53-Arresten interaction elevated above normal.

Expected or normal levels of Arresten expression for a particular diagnostic sample taken from a subject or patient may be easily determined by assaying non-diseased subjects of a similar age and of the same gender. For example, diagnostic samples may be obtained from at least 30 normal, healthy women within a certain age range (e.g., between the ages of 25 and 80), to determine the normal quantity of Arresten expression in females. Once the necessary or desired samples have been obtained, the normal levels of Arresten expression in women may be determined using a standard assay for quantification, such as flow cytometry, Western blot analysis, or an ELISA for measuring protein quantities.

By way of example, an ELISA may be run on each sample in duplicate, and the means and standard deviations of the quantity of the Arresten molecule may be determined. If necessary, additional subjects may be recruited before the normal quantities of Arresten expression are quantified. A similar type of procedure may be used to determine expected or normal levels of p53-Arresten interaction for a particular diagnostic sample taken from a subject or patient.

Immunoassay Detecting Arresten

Immunoassays rely on antigen-antibody interactions, in which an antibody inherently binds to the specific structure of its associated molecule. Conventional immunoassay protocols may be adapted to detect Arresten as a biomarker to diagnose ovarian cancer in a subject. Suitable immunoassay methods include quantitative or qualitative immunoassay methods conventionally developed, including Western blotting (WB), immunofluorescence (IF), immunocytochemistry (ICC), immunohistochemistry immunoprecipitation (IP), enzyme-linked immunosorbent assay (ELISA), and flow cytometry (FCM).

All immunoassays employ a means to produce a measurable signal in response to antibody binding, and most involve chemically linking a detectable label to antibodies or antigens. Many labels are detectable because, for example, they emit radiation, produce a color change in a solution, fluoresce under light, or can be induced to emit light.

Production of Anti-Arresten Antibody

In certain embodiments of the present invention, the agent reactive with Arresten is an antibody. Such an antibody may be produced using conventional methods known in the art, and may be polyclonal or monoclonal.

Polyclonal antibody may be produced, for example, by immunizing any animal species host, such as a rabbit, mouse, rat, sheep, goat, monkey, and the like, with purified Arresten. Monoclonal antibody then may be produced by removing the spleen from the immunized animal, and fusing the spleen cells with myeloma cells to form a hybridoma which, when grown in culture, will produce a monoclonal antibody (see, e.g., Kohler and Milstein [53]). Alternatively, a phage antibody library (see, e.g., Clackson et al. [54]; Marks et al. [55]) or any other methods known in the art may be used to produce monoclonal antibody. The antibodies of the present invention include functional fragments of antibody molecules. A “functional fragment” of an antibody is an antibody fragment having antigen-binding function.

In one embodiment, a diagnostic tool of the present invention uses, as a reagent, a monoclonal antibody specific to Arresten. Such an antibody can be produced by the following method: (i) polyclonal antibody is produced by immunizing a rabbit with purified human Arresten; (ii) monoclonal antibody is produced by removing the spleen from the immunized rabbit, and fusing the spleen cells with myeloma cells to form a hybridoma; and (iii) the hybridoma is grown in culture and the monoclonal antibody is isolated.

Tests and Kits

The inventor's determination that an elevated level of Arresten expression can be detected in diagnostic samples from ovarian cancer patients provides new options for identifying patients with ovarian cancer, with the potential for commercial application in the form of diagnostic tools (e.g., tests, kits, etc.) for the diagnosis of ovarian cancer and for use in general screening procedures. Screening procedures can assist in the early detection and diagnosis of ovarian cancers, and can provide a method for the follow-up of patients in whom Arresten expression elevated above normal has been detected.

Accordingly, the present invention further provides tests and kits for use as assays for ovarian cancer. In some embodiments, the test or kit includes an agent reactive with Arresten and reagents suitable for detecting expression of Arresten. The agent may be any of those described above, and may be used in any of the above-described assays or methods for detecting or quantifying Arresten expression. Preferably, the agent of the present invention is labeled with a detectable marker or label.

As noted above, ELISA is one of several immunoassay methods used to detect and quantify specific target molecules. Three common types of ELISA are sandwich assays, competitive assays, and antigen-down assays. Sandwich assays are most commonly used because they deliver more sensitive and robust results.

In one embodiment, the diagnostic tool of the present invention is a sandwich ELISA capable of detecting Arresten in biological samples (e.g., blood, plasma, urine, etc.). By way of example, such a sandwich ELISA may include the following steps: (i) coating an antibody capable of binding to an epitope of Arresten, for use as a capture antibody attached to the surface of a solid substrate; (ii) reacting the capture antibody with the biological sample; (iii) reacting the resultant product of step (ii) with a detection antibody that is capable of binding to the complex of Arresten and the capture antibody and is labeled with a signal generating label; and (iv) measuring a signal originating from the label. The signal outputs can be measured in accordance with various methods known in the art. This detection of the signal enables a qualitative or quantitative analysis of the biomarker. If biotin is used as a label, it can be easily detected by streptavidin. When luciferase is used, luciferin can easily detect a signal. By analyzing the intensity of the final signal by the above-described immunoassay, ovarian cancer can be diagnosed. Specifically, when the signal for the biomarker in the biological sample of a subject appears stronger than in the normal sample, it can be determined that the subject has ovarian cancer.

In another embodiment, the diagnostic tool of the present invention is a compact point-of-care (POC) instrument employing ELISA for measuring Arresten in blood, plasma, serum, urine, and the like. The ELISA-based point-of-care (POC) testing may be suitable for use in resource-limited settings (e.g., a clinician's office) without requiring patients to visit a specialized laboratory for testing. The POC testing may adopt principles such as simplification of the procedures and miniaturization of the testing devices. Such a POC platform may, for example, combine the process of sandwich ELISA and the readout into a single microfluidic chip or cartridge, thereby providing a fully integrated, instrument-free, low-cost, and portable device for OC screening. Antibodies with relatively high affinity to Arresten may be preferred for such POC testing methods.

Due to its easy access, a POC test for ovarian cancer can be more readily available for screening of women with any relevant symptoms. Especially when symptoms are vague at early stages of the disease, availability of such convenient diagnostic tools may change the behaviors of clinicians and their patients, and result in promotion of routine screening of the population for ovarian cancer.

In certain embodiments, POC technology is combined with a urine test. Urine testing is particularly advantageous because urine samples are easily available and can be collected frequently in a non-invasive way. Correlation between urine and blood levels of Arresten can demonstrate the usefulness of urine detection of Arresten as a surrogate for detection of Arresten in plasma.

By way of example, a subject's urine sample may be screened for Arresten levels as part of a routine health examination. The test may be formatted as a urine test strip (e.g., as a standalone strip, cassette, or dipstick for laboratory use). A urine test strip for identifying whether a subject is at risk of having or has ovarian cancer may comprise a reagent that provides a response to the presence of Arresten when immersed in, and removed from, a urine sample of the subject; such a response indicates whether the subject is at risk of having or has ovarian cancer.

The urine test strip may be produced by means known to a person skilled in the art. In one embodiment, the urine test strip is provided in a device for testing a urine sample of a subject. The urine test strip may be prepared with a pad or matrix of an absorbent material. A labeled antibody specific to Arresten may also be provided in an area on the urine strip, so that urine, the labeled antibody, and Arresten flow together through the absorbent material by capillary action. The strip can be prepared from any suitable material through which the urine test sample, any Arresten therein, and labeled antibody can flow by capillarity. Suitable matrix materials include, without limitation, nitrocellulose, polysulfones, polycarboxylic acids, and filter paper.

In another embodiment, a urine test device may contain an immunoassay strip, an absorbent matrix or pad, and a plastic housing. The immunoassay strip can be formed by compressing nonwoven fibers into a narrow strip and coating them at least partially with a reactive antibody. In use, the antibody of the device may bind to Arresten present in a urine sample, ultimately resulting in a color change. The absorbent pad or matrix may extend to contact the urine stream of a user. The absorbent matrix can absorb the liquid and draw it into contact with the immunoassay strip. The immunoassay strip and the absorbent pad may be contained within a 2-piece housing that allows the unit to be handheld and protects the strip from environmental contaminants. A leak-proof, clear plastic window on the side of the housing can prevent urine from accidentally splashing on the immunoassay strip and permit the test and control zone portions of the strip to be viewed.

In still another embodiment, a strip-based ELISA test, similar to a pregnancy test, can be used by individual women at their own homes to detect Arresten in urine samples. This can promote ovarian cancer screening for even asymptomatic women, obviating the need to go to a doctor's office or laboratory for testing.

As noted above, ovarian cancers are associated with high fatality, but early detection can significantly improve survival rates. Convenient urine tests for use in detecting Arresten as a biomarker in ovarian cancer may promote early detection in patients, thereby increasing survival rates.

Exemplary Methods

In accordance the present invention, Arresten as an ovarian cancer biomarker may be detected, alone or in combination with other markers such as CA125 and HE4, in a biological sample. As discussed above, the cut-off value for CA125 may be increased (e.g., to 95-100 U/ml) in order to increase sensitivity of the biomarker for late-stage ovarian cancer (e.g., stage III or IV), but at the expense of detecting patients with early stage ovarian cancer (e.g., stage I or II). Combining Arresten detection with CA125 detection (with an increased cut-off value) may compensate for detecting stage I/II patients. Therefore, in one embodiment, a diagnostic sample of a subject is assayed for detecting the expression of both Arresten and CA125 to diagnose ovarian cancer in the subject. In certain preferred embodiments, the cut-off value for CA125 is 80-120 kU/L, 85-110 kU/L, 90-105 kU/L, or 95-100 kU/L. More preferably, the cut-off value for CA125 is 95 kU/L or 100 kU/L.

A small proportion of ovarian cancer patients do not have defective p53 and, therefore, may not show a high level of Arresten expression. Such non-mutated p53 ovarian cancers have a better prognosis in general. Accordingly, the present invention also provides a method for determining the molecular subtype (type I or II) of ovarian cancer and/or assessing the severity of the disease in a subject or patient who has been diagnosed with ovarian cancer. For example, if the assay of a biological sample from a patient who has been diagnosed with ovarian cancer does not indicate Arresten expression elevated above normal, it may be determined that the patient has type I ovarian cancer with a good prognosis (i.e., positive prognosis). If the assay of a biological sample from a patient who has been diagnosed with ovarian cancer indicates Arresten expression elevated above normal, it may be determined that the patient has type II ovarian cancer with a poor prognosis (i.e., negative prognosis).

The present invention further provides a method for assessing the efficacy of therapy to treat ovarian cancer in a subject or patient who has undergone or is undergoing treatment for ovarian cancer. The method includes assaying a diagnostic sample of the subject or patient for Arresten expression, where detection of a normal level of Arresten expression is indicative of successful therapy to treat ovarian cancer, and detection of Arresten expression elevated above normal is indicative of not responding to treatment. In one embodiment of the method, Arresten expression elevated above normal is detected by detecting p53-Arresten interaction elevated above normal. The ovarian cancer may be any of those described above, including p53-associated ovarian cancer. The diagnostic sample may be a tissue or a bodily fluid, as described above, and may be assayed for expression of Arresten. In addition, the diagnostic sample may be assayed for expression of Arresten using all of the various assays and methods of detection and quantification described above.

This method of the present invention provides a means for monitoring the effectiveness of therapy to treat ovarian cancer by permitting the periodic assessment of levels of Arresten expression in a diagnostic sample taken from a subject or patient. In accordance with this method, a diagnostic sample of a subject or patient may be assayed, and levels of Arresten expression may be assessed, at any time following the initiation of therapy to treat ovarian cancer. For example, levels of Arresten expression may be assessed while the subject or patient is still undergoing treatment for ovarian cancer. Where levels of Arresten expression detected in an assayed diagnostic sample of the subject or patient continue to remain elevated above normal, a physician may choose to continue with the subject's or patient's treatment for the cancer or change it to a treatment to which tumour cells better respond. Where levels of Arresten expression in an assayed diagnostic sample of the subject or patient decrease through successive assessments, it may be an indication that the treatment for ovarian cancer is working, and that treatment doses could be decreased or even ceased. Where levels of Arresten expression in an assayed diagnostic sample of the subject or patient do not rapidly decrease through successive assessments, it may be an indication that the treatment for ovarian cancer is not working, and that treatment doses could be increased or the choice of treatment could be changed.

It is within the confines of the present invention to assess levels of Arresten expression following completion of a subject's or patient's treatment for ovarian cancer, in order to determine whether the cancer has recurred in the subject or patient. Accordingly, an assessment of levels of Arresten expression in an assayed diagnostic sample may provide a convenient way to conduct follow-ups of patients who were previously diagnosed with ovarian cancer. Furthermore, it is within the confines of the present invention to use assessed levels of Arresten expression in an assayed diagnostic sample as a clinical or pathologic staging tool, as a means of determining the extent of ovarian cancer in the subject or patient, and as a means of ascertaining appropriate treatment options.

A correlation exists, in general, between tumour burden and the survival of a patient who has cancer, and, more specifically, between pelvic mass and an ovarian cancer patient. Therefore, it is also contemplated herein that assaying a diagnostic sample of a subject for Arresten expression may be a useful means of providing information concerning the prognosis of a subject or patient who has ovarian cancer. Accordingly, the present invention further provides a method for assessing the prognosis of a subject who has ovarian cancer, comprising assaying a diagnostic sample of the subject for Arresten expression, where the subject's prognosis improves with a decrease in Arresten expression in the diagnostic sample of the subject, and the subject's prognosis worsens with an increase in Arresten expression in the diagnostic sample of the subject. Persistent high-level Arresten means that ovarian cells with dysfunctional p53 still exist.

According to the method of the present invention, a diagnostic sample of a subject or patient may be assayed, and levels of Arresten expression may be assessed, at any time during or following the diagnosis of ovarian cancer in the subject or patient. For example, levels of Arresten expression in an assayed diagnostic sample may be assessed before the subject or patient undergoes treatment for ovarian cancer, in order to determine the subject's or patient's initial prognosis. Additionally, levels of Arresten expression in an assayed diagnostic sample may be assessed while the subject or patient is undergoing treatment for ovarian cancer, in order to determine whether the subject's or patient's prognosis has become more or less favorable through the course of treatment.

By way of example, where levels of Arresten expression detected in an assayed diagnostic sample of the subject or patient are, or continue to remain, significantly high, a physician may conclude that the subject's or patient's prognosis is unfavorable. Where Arresten expression in an assayed diagnostic sample of the subject or patient decreases through successive assessments, it may be an indication that the subject's or patient's prognosis is improving. Where levels of Arresten expression in an assayed diagnostic sample of the subject or patient do not decrease significantly through successive assessments, it may be an indication that the subject's or patient's prognosis is not improving. Finally, where Arresten expression is low, or is normal, in a diagnostic sample of the subject or patient, a physician may conclude that the subject's or patient's prognosis is favorable.

The present invention also provides methods of treating a subject who has been diagnosed with ovarian cancer using Arresten as a biomarker. The standard treatment for ovarian cancer currently consists of debulking surgery followed by six rounds of chemotherapy. In one embodiment, a diagnostic sample of a patient is analyzed for Arresten expression, and a therapy is provided to the subject when the Arresten expression is elevated above normal. Exemplary therapies include, without limitation, surgical debulking, chemotherapy, radiation, or hormonal therapy, and any combination of the foregoing, including the current standard treatment of debulking surgery followed by chemotherapy. Typical chemotherapy drugs combine a platinum-based drug (e.g., carboplatin or cisplatin) with a taxane (e.g., paclitaxel or docetaxel).

The present invention is described in the following Examples, which are set forth to aid in an understanding of the invention, and should not be construed to limit in any way the scope of the invention as defined in the claims which follow thereafter.

### EXAMPLES

### Example 1—Study Subjects

Plasma samples derived from ovarian cancer patients (n=22) and healthy controls (n=20) were tested for Arresten levels by an enzyme-linked immunosorbent assay (ELISA).

The ovarian cancer cases were obtained from the Poland Ovarian Cancer Study (POC). The POC study consisted of women with familial ovarian cancer who were referred to 1 of 16 clinical cancer genetics centers throughout Poland between 1999 and 2001. These centers were established in 1998 as a national network with support from the Polish Ministry of Health for the purpose of coordinating cancer genetics services. Eligible women included those with invasive ovarian cancer and at least one first- or second-degree relative with ovarian cancer diagnosed at any age or with early-onset breast cancer (diagnosed at age 50 or below). Of those, 22 ovarian cancer patients with high-grade serous type were randomly selected for this study before they started treatment.

The control women were those who participated in mammography screening for their routine screening tests at 8 different centers all over Poland between 2009 and 2011 and who provided blood samples for DNA analysis. Women with breast or ovarian cancer were excluded from this group; 20 healthy women were randomly selected for the study.

### Example 2—Laboratory Test

Plasma Arresten was quantified using a laboratory-made ELISA assay. All plasma samples were run in duplicate. The Arresten concentration (ng/ml) was calculated as the average of duplicate samples (each adjusted for background signal and normalized to blank wells). The average intra-assay coefficient of variation (CV) was approximately 1.7%. This was calculated using the mean CV of duplicate samples. A non-parametric Mann-Whitney U test was used for comparing the mean Arresten plasma levels in plasma of the cases and the controls.

### Example 3—Detection of Arresten in Urine Samples

The same ELISA method of Example 2 that was used for detecting Arresten levels in plasma was also used to measure Arresten in urine samples of four healthy individuals. It was shown that Arresten is detectable in urine and its urine level generally correlates with the matched plasma level.

A urine-based biomarker test facilitates screening and diagnosis processes due to non-invasiveness of the collection method and the feasibility of developing ELISA paper strips to be used by women periodically for testing for ovarian cancer in the comfort of their own homes.

### Example 4—Validation Study

400 plasma samples are collected from ovarian cancer patients with different histology, and 400 control samples collected from healthy subjects. Ovarian cancer plasma samples are obtained from Princess Margaret Hospital's Ovarian Cancer Biobank in Toronto, Canada. The control plasma samples are obtained from the biobank at Women's College Hospital in Toronto. From each patient, 300 μl of plasma is used for measuring three biomarkers: Arresten, HE4, and CA125. Three sections (10 microns each) from ovarian-tumour formalin-fixed paraffin-embedded (FFPE) blocks are obtained from 20 randomly-selected patients for measuring p53 and Arresten expression in tumour tissues.

Arresten levels are determined by enzyme-linked immunosorbent assay (ELISA). The specificity and sensitivity of Arresten as a biomarker are determined, alone and in combination with HE4 and CA-125—other known biomarkers which are also measured by ELISA.

The expression levels of Arresten and p53 in ovarian tumour tissues are measured by immunohistochemistry (IHC) assay.

In order to determine diagnostic performance and optimal cut-off values for Arresten plasma levels, the sensitivities, specificities, and area under a receiver operating characteristic curve (AUC) are calculated. Similar calculations are performed for CA125 and HE4 and for combined biomarkers. The significance of two-group comparisons are calculated using a Mann-Whitney non-parametric test. Biomarker composite scores are calculated by logistic regression from standardized biomarker values.

The power of this study is more than 90% to detect a 10% difference in mean of measured biomarkers between patient cases and controls, based on 400 samples in each group.

### RESULTS AND DISCUSSION

The results obtained from Examples 1-2 are summarized and discussed below.

As shown in FIG. 1, the mean plasma level of Arresten was 573 ng/ml higher in ovarian cancer patients than in healthy controls (1020 versus 446; p=0.0001). This represents a two-fold higher plasma level among ovarian cancer patients, which is opposite to what was expected. The higher concentration of Arresten in the plasma of ovarian cancer patients makes this protein even more favorable as a screening or diagnostic biomarker, despite the contrary hypothesis that linked Arresten with defective p53 function in ovarian cancers. One explanation for the contrary observation is that Arresten is expected to be triggered by high expression levels of p53 even in its mutated, non-functional form. Preferably, it is determined whether ovarian cancer patients tested here also carry high expression levels of the mutant p53 protein.

### REFERENCES

1. Siegel, R. et al., Cancer statistics. CA Cancer J Clin, 2012. 62:10-29.
2. Canadian Cancer Statistics, 2016.
3. Scully, R. E. et al., Histological typing of ovarian tumours. Vol. 9. New York: Springer Berlin; 1999.
4. Torre, L. A. et al., Global cancer statistics, 2012. CA Cancer J Clin, 2015. 65:87-108.
5. Visintin, I. et al., Diagnostic markers for early detection of ovarian cancer. Clin Cancer Res, 2008. 14:1065-1072.
6. Beesley, V. L. et al., Quality of life and treatment response among women with platinum-resistant versus platinum-sensitive ovarian cancer treated for progression: a prospective analysis. Gynecol Oncol, 2014. 132:130-136.
7. Piver, M. S. et al., Five-year survival for stage IC or stage I grade 3 epithelial ovarian cancer treated with cisplatin-based chemotherapy. Gynecol Oncol, 1992. 46:357-360.
8. Jemal, A. et al., Cancer statistics. CA Cancer J Clin. 2010. 60:277-300.
9. Berkenblit, A. et al., Advances in the management of epithelial ovarian cancer. J Reprod Med, 2005. 50:426-438.
10, Soletormos, G. et al., Clinical use of cancer biomarkers in epithelial ovarian cancer-updated guidelines from the European Group on Tumour Markers. Int J Gynecol Canc, 2016. 26(1):43-51.
11. Sturgeon, C. M. et al., National Academy of Clinical Biochemistry laboratory medicine practice guidelines for use of tumour markers in testicular, prostate, colorectal, breast, and ovarian cancers. Clin Chem, 2008. 54:e11-e79.
12. Van Altena, A. M. et al., CA125 nadir concentration is an independent predictor of tumour recurrence in patients with ovarian cancer: a population-based study. Gynecol Oncol, 2010. 119:265-269.
13. Zurawski Je, V. R. et al., An initial analysis of preoperative serum CA125 levels in patients with early stage ovarian carcinoma. Gynecol Oncol. 1988. 30:7-14.
14. Duffy, M. J. et al., CA125 in ovarian cancer: European Group on Tumour Markers. Int J Gynecol Canc, 2005. 15:679-691.
15. Liu, J. et al., Anti-angiogenic agents in ovarian cancer: dawn of a new era? Curr Oncol Rep, 2011. 13:450-458.
16. Schummer, M. et al., Comparative hybridization of an array of 21500 ovarian cDNAs for the discovery of genes overexpressed in ovarian carcinomas. Gene, 1999. 238:375-385.
17. Moor, R. G. et al., Serum HE4 levels are less frequently elevated than CA125 in women with benign gynecologic disorders. Am J Obstet Gynecol, 2014. 206(4):351.e1-351.e8.
18. Li, J., et al., HE4 as a biomarker for ovarian and endometrial cancer management. Expert Rev Mol Diagn. 2009. 9(6):555-566.
19. Moor, R. G. et al., The use of multiple novel tumour biomarkers for the detection of ovarian carcinoma in patients with pelvic mass. Gynecol Oncol, 2008. 108(2):402-408.
20. Molina, R. et al., HE4 a novel tumour marker for ovarian cancer: comparison with CA125 and ROMA algorithm patients with gynecological diseases. Tumour Biol, 2011. 32:1087-1095.
21. Zheng, H. et al., Serum HE4 as a useful biomarker in discriminating ovarian cancer from benign pelvic disease. Int J Gynecol Cancer. 2012. 22:1000-1005.
22. Drapkin, R. et al., Human epididymis protein 4 (HE4) is a secreted glycoprotein that is overexpressed by serous and endometrioid ovarian carcinomas. Cancer Res, 2005. 65(6):2162-2169.
23, Simmons, A. R. et al., The emerging role of HE4 in the evaluation of epithelial ovarian and endometrial carcinomas. Oncology (Williston Park), 2013. 27:548-556.
24. Moor, R. G. et al., A novel multiple biomarker assay utilizing HE4 and CA125 for the prediction of ovarian cancer in patients with pelvic mass. Gynecol Oncol, 2009. 112:40-46.
25. Van Gorp, T. et al., HE4 and CA125 as a diagnostic test in ovarian cancer: prospective validation of the Risk of Ovarian malignancy Algorithm. Br J Cancer, 2011. 104:863-870.
26. Dayyani, F. et al., Diagnostic performance of risk of ovarian malignancy algorithm against CA125 and HE4 in connection with ovarian cancer. Int J Gynecol Canc, 2016.
27. Wang, J. et al., Diagnostic accuracy of serum HE4, CA125 and ROMA in patients with ovarian cancer: a meta-analysis. Tumour Biology, 2014. 35(6):6127-6138.
28. Montagnana, M. et al., The ROMA (Risk of Ovarian malignancy Algorithm) for estimating the risk of epithelial ovarian cancer in women presenting with pelvic mass: is it really useful? Clin Chem Lab Med, 2011. 49:521-525.
29. Karlsen, M. A. et al., Evaluation of HE4, CA125, risk of ovarian malignancy algorithm (ROMA) and risk of malignancy index (RMI) as diagnostic tools of epithelial ovarian cancer in patients with pelvic mass. Gynecol Oncol, 2012. 127:379-383.
30. Ueland, F. R. et al., A perspective on ovarian cancer biomarkers: past, present and yet-to-come, Diagnostics, 2017. 7-14.
31. Soussi, T. & Wiman, K. G., Shaping genetic alterations in human cancer: the p53 mutation paradigm. Cancer Cell, 2007. 12:303-312.
32. Kandoth, C. et al., Mutational landscape and significance across 12 major cancer types. Nature, 2013. 502:333-339.
33. Bieging, K. T. et al., Unravelling mechanisms of p53-mediated tumour suppression. Nat Rev Cancer, 2014. 14:359-370.
34. Bieging, K. T. et al., Unravelling mechanisms of p53-mediated tumour suppression. Nat Rev Cancer, 2014. 14:359-370.
35, Oren, M. & Rotter, V., Mutant p53 gain-of-function in cancer. Cold Spring Harb Perspect Biol, 2010. 2:a001107.
36. Leroy, B. et al., The TP53 website: an integrative resource centre for the TP53 mutation database and TP53 mutant analysis. Nucleic Acids Res, 2013. 41:D962-969.
37. Hahn, W. C. et al., Modelling the molecular circuitry of cancer. Nat Rev Cancer, 2002. 2:331-341.
38, Sherr, C. J. et al., The RB and p53 pathways in cancer. Cancer Cell, 2002. 2:103-112.
39. Kusume, T. et al., The p16-cyclin D1/CDK4-pRb pathway and clinical outcome in epithelial ovarian cancer. Clin Cancer Res. 1999. 5:4152-4157.
40. Fujita, M. et al., Alteration of p16 and p15 genes in common epithelial ovarian tumours. Int J Cancer. 1997. 74:148-155.
41. Katsaros, D. et al., Methylation of tumour suppressor gene p16 and prognosis of epithelial ovarian cancer. Gynecol Oncol. 2004. 94:685-692.
42. Colorado, P. C. et al., Anti-angiogenic cues from vascular basement membrane collagen, Cancer Res, 2000. 60:2520-2526.
43, Nyberg, P. et al., Characterization of the anti-angiogenic properties of arresten, an al β1 integrin-dependent collagen-derived tumour suppressor. Exp Cell Res, 2008. 314:3292-3305.
44. Teodoro, J. G., et al., p53-mediated inhibition of angiogenesis through up-regulation of a collagen prolyl hydroxylase. Science, 2006. 313(5789):968-71.
45. Assadian, S., et al., p53 inhibits angiogenesis by inducing the production of Arresten. Cancer Res., 2012. 72(5):1270-9.
46. Bell, D. et al., Integrated genomic analyses of ovarian carcinoma. Nature, 2011. 474:609-615.
47. Patch, A. M. et al., Whole-genome characterization of chemoresistant ovarian cancer. Nature, 2015. 521:489-494.
48. Rosen, B. et al., The impacts of neoadjuvant chemotherapy and of debulking surgery on survival from advanced ovarian cancer. Gynecol Oncol, 2014. 134(3): 462-7.
49. Chi, D. S. et al., An analysis of patients with bulky advanced stage ovarian, tubal, and peritoneal carcinoma treated with primary debulking surgery (PDS) during an identical time period as the randomized EORTC-NCIC trial of PDS vs neoadjuvant chemotherapy (NACT). Gynecol Oncol, 2012. 124(1):10-4.
50. Bast, R. C., Jr. et al., A radioimmunoassay using a monoclonal antibody to monitor the course of epithelial ovarian cancer. N Engl J Med, 1983. 309(15):883-7.
51. Van Calster, B. et al., A novel approach to predict the likelihood of specific ovarian tumour pathology based on serum CA-125: a multicenter observational study. Cancer Epidemiol Biomarkers Prev, 2011. 20(11):2420-8.
52. Bonfrer, J. M. et al., Clinical evaluation of the Byk LIA-mat CA125 II assay: discussion of a reference value. Clin Chem, 1997. 43(3):491-7.
53, Kohler, G. and Milstein, C., European Journal of Immunology, 1976. 6:511-519.
54. Clackson, T. et al. Nature, 1991. 352(6336):624-628.
55. Marks, J. D. et al., J. Mol. Biol., 1991. 222(3):581-597.

All publications mentioned herein are hereby incorporated by reference in their entireties. While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A method for determining whether a subject has ovarian cancer, comprising assaying a diagnostic sample of the subject for Arresten expression, wherein detection of Arresten expression elevated above normal is diagnostic of ovarian cancer in the subject.

2. The method of claim 1, wherein Arresten expression elevated above normal is detected by detecting p53-Arresten interaction elevated above normal.

3. The method of claim 1, further comprising the step of obtaining the diagnostic sample from the subject.

4. The method of claim 1, wherein the ovarian cancer is an epithelial carcinoma.

5. The method of claim 4, wherein the epithelial carcinoma is a serous, mucinous, endometrioid, clear cell, transitional cell, squamous cell, or mixed epithelial neoplasm.

6. The method of claim 1, wherein the ovarian cancer is a p53-associated ovarian cancer.

7. The method of claim 1, wherein the ovarian cancer is primary ovarian cancer.

8. The method of claim 1, wherein the ovarian cancer is high-grade serous ovarian cancer.

9. The method of claim 1, wherein the diagnostic sample is a serum, plasma, or urine sample.

10. The method of claim 1, wherein the diagnostic sample is assayed using an agent reactive with Arresten.

11. The method of claim 10, wherein the agent is an antibody or an antigen-binding fragment thereof.

12. The method of claim 10 or 11, wherein the agent is labeled with a detectable marker.

13. The method of claim 1, wherein the diagnostic sample is assayed using an ELISA, a chemiluminescence assay, or an immunohistochemistry assay.

14. The method of claim 13, wherein the ELISA is an ELISA paper strip.

15. The method of claim 1, wherein the detected Arresten expression is at least two times higher than normal.

16. The method of claim 1, wherein the Arresten expression is elevated above normal in the diagnostic sample when the Arresten expression is above a concentration selected from 400 to 1000 ng/ml.

17. The method of claim 16, wherein the diagnostic sample is plasma.

18. The method of claim 1, wherein the subject is at least one of the following:
(a) pre-menopausal;
(b) asymptomatic of ovarian cancer;
(c) not carrying a mutation of BRCA1 or BRCA2 gene;
(d) suffering from a non-malignant gynecologic disease, a peritoneal, pleural, or musculoskeletal inflammatory disorder, a pelvic inflammatory disease, a liver, renal, or cardiac disease, or an advanced adenocarcinoma.

19. The method of claim 1, wherein the ovarian cancer is a type II ovarian cancer.

20. The method of claim 19, wherein the type II ovarian cancer is at stage I, II, IIIa, or IIIb.

21. The method of claim 1, wherein the ovarian cancer is undetectable by a CA125 test or a transvaginal ultrasound test.

22. The method of claim 1, further comprising assaying the diagnostic sample of the subject for at least one additional biomarker selected from CA125 and HE4.

23. The method of claim 22, wherein the at least one additional biomarker is CA125 with a cut-off value of 95-100 U/ml.

24. A method of screening a general population for ovarian cancer, comprising: testing a plurality of asymptomatic subjects in accordance with the method of claim 1.

25-44. (canceled)
